(19) United States
(12) Patent Application Publication
(10) Pub. No.: US 2005/0003530 A1
(43) Pub. Date: Jan. 6, 2005

(54) Bioreactor for neuronal cells and hybrid neuronal cell systems

(76) Inventor: Joerg C. Gerlach, Pittsburgh, PA (US)

Correspondence Address: Joerg C. Gerlach MD, PhD, 3613 Butler St, Pittsburgh, PA, 15201, US

(21) Appl. No.: 10/866,124

(22) Filed: Jun. 12, 2004

(30) Foreign Application Priority Data
Jun. 13, 2003 (DE) 103 26 748.4

(51) Int. Cl. C12N 5/08; C12M 1/00

(52) U.S. Cl. 435/368; 435/293.1

(57) ABSTRACT

The invention applies to a bioreactor for neuronal cell cultures and neuronal cell culture systems with an arrangement of such neuronal cell bioreactors and their application thereof. Neuronal cell culture bioreactors are especially required in the areas of medicine and research in natural sciences. The purpose of this invention is to facilitate a 3-dimensional (3D) arrangement of neuronal cells in a bioreactor as well as a network of neuronal cells across various bioreactors.

The invention applies to a bioreactor for neuronal cell cultures and neuronal cell culture systems with an arrangement of such neuronal cell bioreactors and their application thereof. Neuronal cell culture bioreactors are especially required in the areas of medicine and research in natural sciences.

Neuronal cells can already be kept alive outside the body (in vitro). At this, conventional culture techniques allow for cell proliferation and preservation of certain characteristics that are typical for neuronal cells.

This includes, for instance, the development of cell projections, which are vital for the development of neuronal networks in the body. Already known, more elaborate culture models, allow for the attachment of neuronal cells to electrical systems, e.g. sensors, effectors, or computer systems to measure or influence cell typical characteristics of signal circuitries (bmp-high-tech, 06/2001, pp. 18-20). Thereby neuronal cells are immobilized on the surface of a chip to prevent them from moving and migrating. This does not hinder the development of cell projections. Additionally, electrodes are attached to the surface of this chip to conduct electrical signals form the neuronal cells that proceed from cell projections. These 2-dimensional (2D) cell culture models for neuronal cells arrange the cells two-dimensionally onto microchips, whereby the cells are able to receive and send signals via the cell projections. Therewith, the typical neuronal functions are already measurable and utilizable for signal processing. Therefore, hybrid in vitro models for neuronal cells are already known in 2D-applications, especially the process of linking effectors, sensors or electrical systems with neuronal cells.

These 2D-cell cultures however are still limited to very basic connections. An in vitro culture of neuronal cells that allows neuronal functions of the brain, spinal cord, connections of nerve tracts (ganglia) and/or the uptake of signals from receptors, as well as the release of signals to effectors, e.g. muscle cells, in a 3D solution is of great interest.

Culture models that mimic neuronal cell functions in more complex arrangements are of interest, not only in fundamental research, but also in applied research in industry, in the development of new therapies, as well as for regenerative medicine R+D.

The purpose of this invention is to facilitate a 3-dimensional (3D) arrangement of neuronal cells in a bioreactor as well as a network of neuronal cells across various bioreactors.

This purpose is achieved via a neuronal cell culture bioreactor according to patent claim 1, and through an arrangement of neuronal cell culture bioreactors according to patent claim 32. Advantageous developments of the neuronal cell culture bioreactor, as well as the arrangements are given in the relative claims.

According to the invention, the neuronal cell culture bioreactor exhibits a container in which neuronal cells settle and are in contact with each other. Furthermore, the neuronal cell culture bioreactor exhibits at least two, independent from each other, channel-like hollow fiber membrane systems (capillary membrane systems), each with a multitude of individual hollow fiber membranes (hollow fibers/capillaries). These capillary membrane systems intersect and superimpose each other, thus creating junctions. At these junctions, outside on the surface of the capillaries, neuronal cells, and neuronal cell conglomerates can settle and facilitate the transmission of signals between the intersecting hollow fibers.

At these intersections the neuronal cells pick up signals, process them, and then forward them to cells of other intersecting hollow fibers. This configuration allows a 3D arrangement of neuronal cells in a bioreactor that largely resembles the natural organization of neuronal cells in the brain, spinal cord, and ganglia, etc. In such 3D spaces, neuronal cells can independently organize themselves, using the aid of co-cultures with glia cells and/or connective tissue cells, to biological neuronal tissue.

As an option a body of open porous material is located inside the casing. The pores of the porous body are in communication with each other, and pathways in the open porous material replace the hollow fibers. These pathways are arranged in a comparable 3D pattern such as the hollow fibers. Through the body's porous surface the neuronal cells settle in this body, whereby they can connect the porous pathways to allow a cell network for communication.

In one embodiment, the hollow fibers of the hollow fiber membrane systems are developed in such a way that they can receive electrical signals from the projections of the neuronal cells and forward them outward of the container and/or release them to projections of other neuronal cells. Hereunto, the hollow fibers have the ability for electrical conduction, and are developed in such a way that they receive and forward electrical signals from neuronal cells. Such circuitry for the transmittal of signals between an electrical system and a neuronal cell has already been described afore (bmp-high-tech, 06/2001, pp. 18-20).

It is advantageous if the hollow fiber membranes exhibit micro pores through which only the outer most ends of the cell projections can penetrate into the lumen of the hollow fibers. Then the reception and transmission can occur through projections from different cells connecting within the lumen of the capillaries while connecting two different hollow fiber membrane systems. Additionally, the conductibility inside the hollow fiber can be improved through electrolyte fluids or gels, gold-or silver wires, as well as other conductors. The conductibility can be improved outside the hollow fibers with electrically conducting material encasing the fibers.

In a second embodiment it is advantageous when the hollow fibers exhibit such pores where switching signals from one bioreactor to another and signal processing can be facilitated via computer systems. The neuronal cells themselves cannot penetrate into the hollow fibers but into their projections. In that case it is possible that the projections inside the hollow fibers increase and extend over several centimeters within the hollow fibers analogous to in vivo nerve cell/nerve. Therewith, the transmission of signals within the hollow fibers via cell projections is possible.

This makes it of interest to switch or propagate signals from one particular hollow fiber membrane, or from one hollow fiber system to another, via neuronal cell projections, during which signal processing can occur. The signals that were switched and transferred via the cell projections to the second hollow fiber membrane system can be conducted out of the bioreactor and directed to another neuronal cell culture bioreactor. In the event that the transmission occurs via cell projections inside the hollow fibers, the circuitry can occur between a projection inside a hollow fiber and a neuronal cell in a neighboring bioreactor. In this case it is advantageous if the hollow fiber membrane systems outside the neuronal cell culture bioreactors exhibit sheathing through which the hollow fiber membrane systems can be infused with nutrients, and oxygen rich medium solutions. This arrangement ensures that the neuronal cell projections, which extend outside the bioreactor, are sufficiently supplied and can adequately dispose of their metabolic waste products.

In the event that the hollow fibers are equipped with an electrical conductor the hollow fiber parts, outlying the neuronal cell culture bioreactor, can be electrically insulated. This imitates the myelin sheath that is naturally present around nerve projections in vivo.

The hollow fiber membrane systems can transverse from outside to inside or vice versa in the neuronal cell bioreactor. If the casing is a cube, e.g. a die, there are six areas available across which up to six hollow fiber membrane systems can enter into or three systems can pass through the respective neuronal cell culture bioreactor. It is also possible, for separate systems of hollow fibers, to enter into the container from two opposite sides or two neighboring sides in which case the hollow fibers run parallel to each other or intersect. Furthermore, via this method, hollow fiber membrane systems can connect two different neuronal cell culture bioreactors.

If the neuronal cells within the neuronal cell culture bioreactor are kept in co-culture with, for example, neuronal connective tissue cells, and/or glia cells, it offers a culture environment very much like the original biological system, which would allow these cells to independently reorganize themselves as biological tissue.

The hollow fiber structures can continue to be utilized for supplementation and waste removal of the cells in the containers. Hereunto, inflow devices can be mounted on the outer side of the container through which either liquid feed or oxygen rich solutions can be applied directly through openings in the container wall or metabolic waste can be removed. Alternatively, the hollow fiber membrane systems can partially lead into the inflow devices, and be perforated in that section to allow the supply of liquid feed for the hollow fiber membrane systems via the inflow devices or the removal of waste product solutions from the bioreactor.

The feeding and oxygenation occurs via inflow and outflow devices on the surrounding casing of the container and/or via hollow fiber structures whose walls are permeable to these agents. Particularly in the latter case a very even distribution of supplying substances, e.g. nutrients, oxygen, electrolytes, mediators, growth factors, hormones, and similar substances is achieved. In this case, the hollow fiber structures inside the container are also porous because porosity facilitates the transfer of supplying substances and the removal of waste products like CO2.

The neuronal cell bioreactor permits a 3D culture system for neuronal cells, which, via the circuitry of their neuronal signals in this 3-dimensional space, can enable the complex functions of a nervous system. In case of an interconnection with other comparable neuronal cell culture bioreactors, the individual neuronal cell culture bioreactors can, for instance, exhibit the functions of spinal cord ganglia that are interconnected and transmit signals from sensors to effectors. The circuitry of information occurs via the neuronal cells, and the information transmission occurs via connecting cell projections of the neuronal cells. Similarly as in the natural body the neuronal projections can extend over several centimeters as so called neurons. The exiting and forwarding of bundled hollow fiber membrane system from a bioreactor allows for the possibility to mimic bundled nerve cell exits from a neuronal cell knot and thereby exhibiting the functions of a neuronal cell strand.

The developed hybrid neuronal cell system preferably consists of individual neuronal cell bioreactors, which allow the circuitry of neuronal cell projections between several bioreactors, analogous to the natural situation.

Characteristic for the development is the appropriate porosity of the hollow fiber structures. In the case of allowing all projections to transverse through the membrane wall, the porous membrane structure may also be further opened by additional mechanical treatments, such as laser hole burning. In the case of allowing all projections to transverse through the membrane wall, the porous membrane structure may also farther opened by additional mechanical treatments, such as laser hole burning.

With pore sizes below the micrometer range, the hollow fiber structures facilitate the transmission of electrical signals through the walls of the hollow fibers, however, avoiding the passage of neuronal cell projections into the hollow fibers. Thereby, the utilization of the cell culture model, restricted to signal processing of neuronal cells, is rendered possible.

This requires that either the hollow fiber structure is electrically conductive, or electrical conductivity is created inside the hollow fiber structure via electrolytes or wires, e.g. made of silver. A special design allows it that the porosity of the membrane walls is designed in such a way that only the very outer most ends of the cell projections can penetrate the porous membrane walls into the lumen of the channels and advance to the electrical conductive structures. Increasing the porosity of the walls of 3D hollow fiber structures to 1-5 micrometer facilitates the interconnection of the neuronal cells inside by allowing the cell projections of different cells to connect within the 3D space of the lumen of the fibers.

Special surface sheathing facilitates the medium perfusion of such structures similar to neuronal strands thereby facilitating the supply of the neuronal cell projections across several centimeters, as well as a connection between several bioreactors.

The possibility of interconnecting several bioreactor systems across above-mentioned connective channels results in a design of complex neuronal networks mimicking different neuronal cell stations in vitro.

Biomatrix protein coatings are often used in cell culture technique but, in this invention, preferably replaced by a co-culture of neuronal cells with neuronal connective tissue; because connective tissue cells, respectively glia cells in neuronal tissue, can produce such a matrix themselves and support the functions of neuronal cells. It is also advantageous to use neuronal stem cells and neuronal progenitor cells, connective tissue progenitor cells, and/or glia progenitor cells. The invention refers to animal and human cells.

The arrangement of the channel like hollow fiber structures inside the neuronal cell culture bioreactor is essential for this invention. If, in a cube shaped container, such channels are arranged in parallel and top to bottom through the bioreactor, the inflow and outflow of signals or cell projections occurs from top to bottom. If, additionally, such structures are arranged front to back and right to left through the bioreactor it is possible to interconnect the signals in such a way that facilitates mathematical modeling of neuronal cell functions to develop artificial intelligence models. Furthermore, models for signal reception and signal processing as well as signal transmission to effector structures are possible. The connection to appropriate sensors and effector structures (through corresponding signal converters) to computers, facilitate mimicking/depiction of neuronal cell networks in vitro.

The invention also allows the conditioning of neuronal cells; especially stem cells/progenitor cells inside the bioreactor.

Therefore, such bioreactors are interesting for the extracorporeal utilization of cells, cell production (for cell transplantation), and as production site for cell products to be applied in regenerative medicine or research.

Via application of mediators, growth factors, hormones, or other effectors, the development, maintenance, preservation, growth, or differentiation of neuronal structures can be facilitated through progenitor cells and cells of the nervous system. Therefore, it is important to not only keep the neuronal cells in co-culture with connective tissue/glia cells but also independently hereof perform a co-culture with progenitor cells. The connective tissue cells and/or further cells may be kept in separate bioreactors, which communicate via mediator liberation into a common medium circuit, and result in the development of self-organized neuronal cell tissue as it occurs in vivo.

The following are descriptions of examples of neuronal cell culture bioreactors.

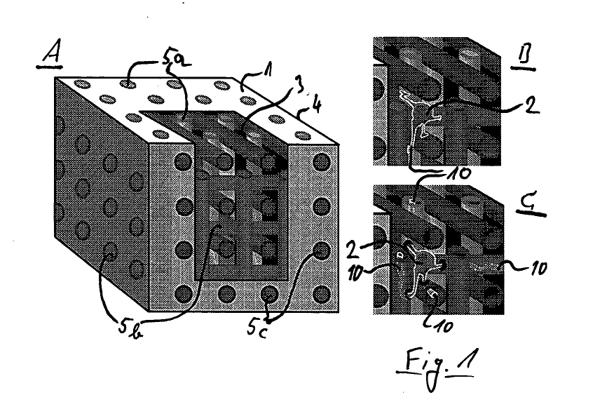

FIG. 1 demonstrates a neuronal cell culture bioreactor

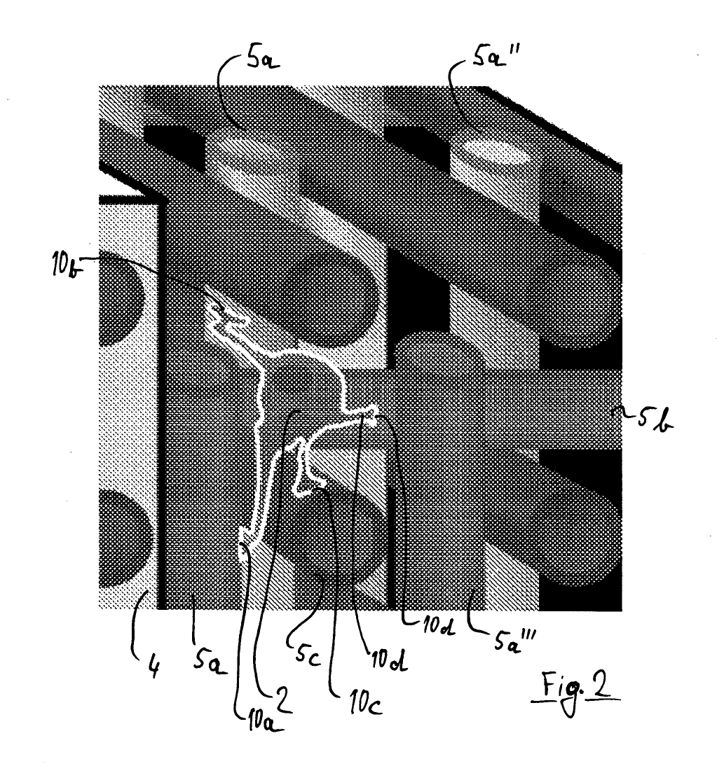

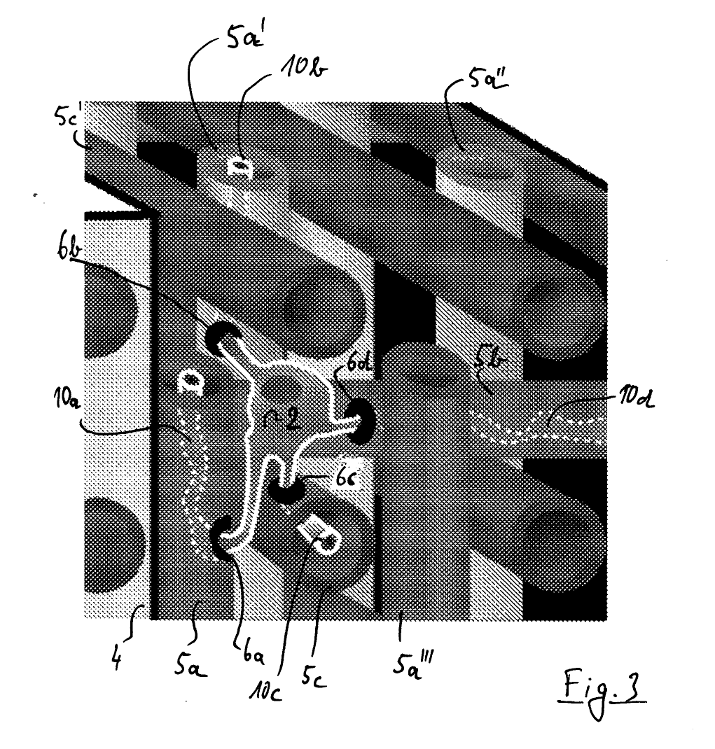

FIG. 2 & FIG. 3 demonstrates sections of the neuronal cell culture bioreactor in FIG. 1

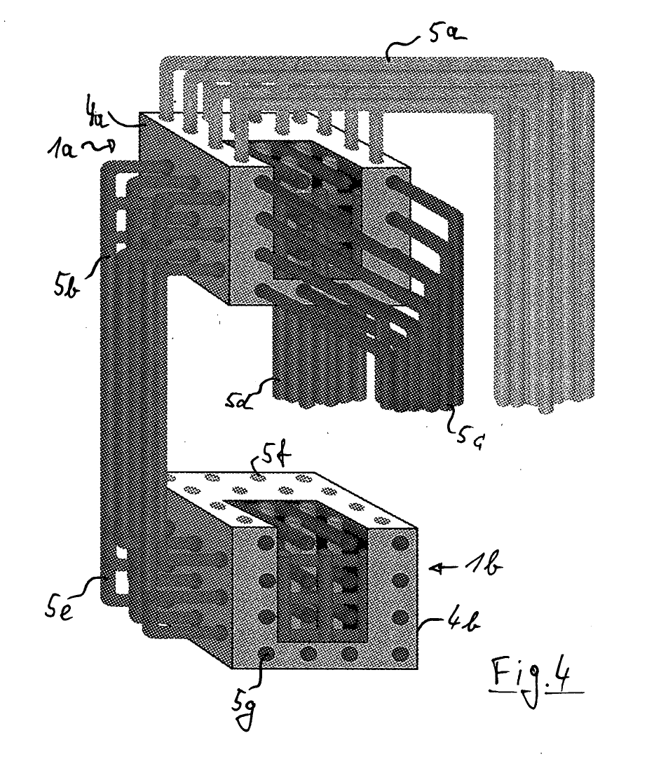

FIG. 4 demonstrates an arrangement of interconnecting neuronal cell culture bioreactors

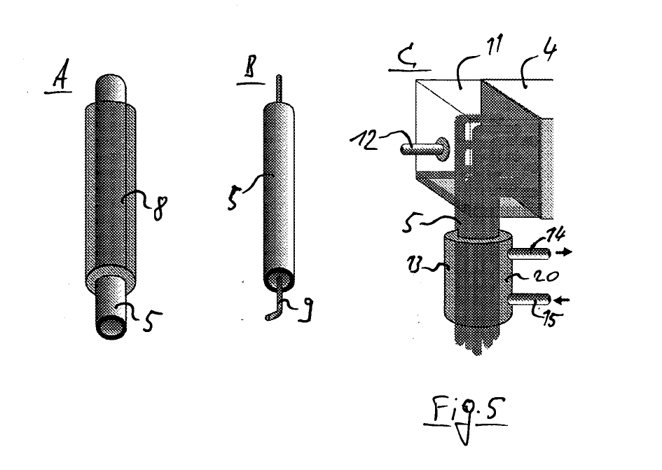

FIG. 5 demonstrates arrangements of the hollow fibers

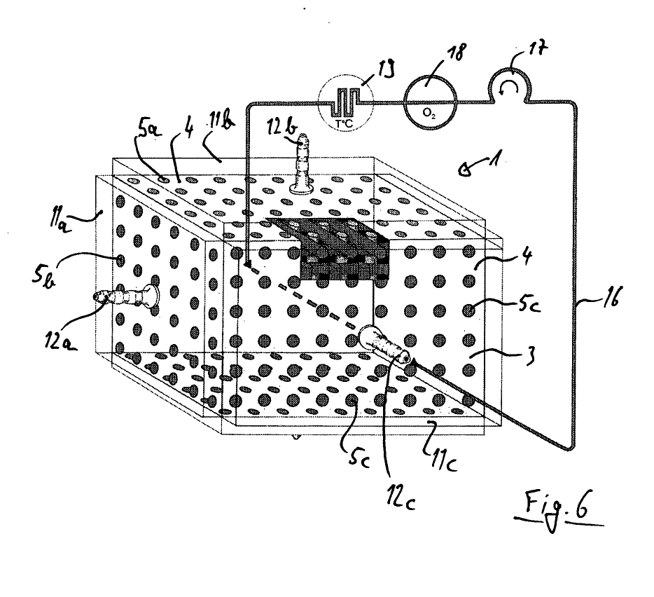

FIG. 6 demonstrates another bioreactor and

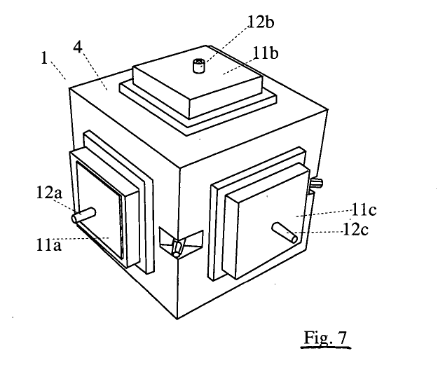

FIG. 7 demonstrates a photograph of a developed neuronal cell culture bioreactor according to FIG. 6. A schematic drawing is included to better demonstrate the features.

FIG. 1 demonstrates a neuronal cell culture bioreactor 1 in chart A. Charts B and C demonstrate enlarged sections from this neuronal cell culture bioreactor. Here, as well as in the following, identical or similar reference signs are used.

The neuronal cell culture bioreactor 1 exhibits an interior chamber 3, in which neuronal cells have settled. These neuronal cells are immobilized on the surface of the capillaries that infuse the body. Inside the cell compartment the body can contain neuronal cells without a scaffold. However, as an option, the body can consist of an open porous sponge like material (not depicted in the illustration), in which the cells are immobilized and which is infused with channel like pathways. The body 3 is surrounded with a cube shaped, respectively dice shaped media-tight container 4. The lateral sides of the container 4 are disrupted by circular openings from which hollow fiber membrane systems 5a, 5b, 5c infuse the body 3. These hollow fiber membrane systems can be developed in varying ways. For example, the walls of the hollow fiber membrane system according to FIG. 1b contain only small porous openings <1 micrometer in diameter so that only the outer most ends of the cell projections 10 of the neuronal cells 2 can penetrate the hollow fiber structures. In FIG. 1c, the walls of the hollow fiber membrane system contain openings through which the neuronal cell projections can infuse the system and grow and expand within.

As recognizable in FIG. 1A, the hollow fiber membrane system can span from one side of the container 4 to the other (e.g. hollow fiber membrane system 5c), or just penetrate the body 3 from outside.

FIG. 2 shows an enlarged version of FIG. 1b. It is plainly visible that the neuronal cell 2 extends its projections 10a through 10d to the hollow fiber membrane systems 5a, 5a′, 5b and 5c, however, only penetrating them with their outer most ends of the projections 10a through 10d. In this case, an interconnection of signals occur between all four hollow fibers 5a, 5a′, 5c, and 5e via the neuronal cell 2. Thereby, a 3D interconnection of the signals is guaranteed between the hollow fibers 5a, 5a′, 5b, 5c for instance via a silver electrode inside each hollow fiber.

FIG. 3 demonstrates an enlarged version of FIG. 1c. In this case the cell projections 10a through 10d extend through the openings 6a through 6b into the hollow fiber membrane systems 5a, 5a′, 5b and 5c for several centimeters. Thus, within the hollow fibers 5a through 5b and via cell projections, the signal can be lead out of the neuronal cell culture bioreactor 1, respectively the container 4.

In the structures of FIG. 1 through 3 the feeding and oxygenation of the neuronal cells occurs in the porous body 3 via the surrounding casing or the hollow fiber structures, whose walls are constructed permeable for certain substances.

FIG. 4 demonstrates an arrangement of two neuronal cell culture bioreactors 1a and 1b of which each one is developed as shown in FIG. 1. The neuronal cell culture bioreactor 1a exhibits four hollow fiber membrane systems 5a through 5d, which can individually be interconnected with effectors, sensors, or another neuronal cell culture bioreactor 11b. In FIG. 4 the neuronal cell culture bioreactor 1b is described with at least three hollow fiber membrane systems (also called hollow pathway systems) 5e, 5f, and 5g. The two hollow fiber membrane systems 5b and 5e are interconnected, to facilitate the transmission of signals between the two neuronal cell culture bioreactors 1a and 1b. Both systems of signal transmission described in FIG. 1b and FIG. 1c, respectively FIGS. 2 and 3 are well suited. The hollow fiber membrane systems 5a through 5f are bundled and lead out of the casing 4a and 4d of the neuronal cell culture bioreactor 1a and 1b, to transmit signals like the biological example of a neuronal strand. Through inward and outward conduction of signals or cell projections form top to bottom, right to left, or front to back, respectively via inward conduction through the surface of the container 4a, 4b, and the outward conduction via a neighboring surface, the neuronal cells have the possibility to interconnect signals in a way that facilitate the mathematical modeling of neuronal cell functions for models of artificial intelligence. The described arrangement can artificially replicate/reproduce whole sections of the nervous system, whereby the bundle of hollow fiber membrane systems outside the container 4a, 4b correspond with the nerve tracts, and the neuronal cell culture bioreactors correspond with the ganglia.

FIG. 5 demonstrates various arrangements of hollow fiber membrane systems. In FIG. 5a the hollow fiber 5 is provided with a coating 8, which is electrically insulated. Such an electrical insulation might be necessary or helpful outside the container if the hollow fiber membrane system itself is electrically conducting, e.g. FIG. 5b, via electrodes (e.g. gold, silver), other conductors, or conductive polymers running inside the hollow fiber 5. Additionally, the electrical insulation is advantageous when cell projections of neuronal cells extend into the hollow fiber 5.

In later case it is advantageous if the hollow fibers of a hollow fiber membrane system 5 are equipped with sheathing 13 that can serve as medium perfusion channel/tube. Hereunto, the sheathing is equipped with an inflow 15 and an outflow 14 through which the hollow fibers 5 can be flushed with media. With appropriate porosity of the hollow fibers it is possible to import nutrients, oxygen, and other substances into the hollow fibers to supply the neuronal cell projections and remove metabolic waste products. In FIG. 5c the container is also equipped with an inflow device 11 that possesses a connection 12 for the supply and removal of media. It is now possible to design a permeable outer wall of the container that exhibits the in/outflow devices 11, so that, through this wall, substances can be infused or removed from the in/outflow devices 11 into of the container 4.

Alternatively, the hollow fibers 5 are permeable so that substances can be infused into the hollow fibers 5 from the inside of the in/outflow device 11 and substances can pass over from the hollow fibers 5 into the inside of the in/outflow devices. Depending on the flow direction of medium inside the hollow fibers 5, the supply or removal of substances to or from the container 4 occurs. The pore size for such porosity is most advantageous at <1 micrometer so substances can enter and leave. However, the neuronal cells cannot grow cell projections from the hollow fibers through these pores.

Through the wall porosity of the hollow fiber structures or the container walls the transfer of nutrients, oxygen supply, the removal of CO2, as well as electrolyte-, pH-, and nutrients homeostasis is possible. This applies analogous to mediators, growth factors, and hormones that can be added to the neuronal cells to also facilitate the maintenance, growth, differentiation or development of neuronal structures from adult stem cells located inside the reactor.

FIG. 6 describes another neuronal cell culture bioreactor 1 with container 4 in which a porous body is arranged. This body 3 is infused with hollow fiber membrane systems 5a, 5b, according to the neuronal cell culture bioreactor in FIG. 1A. This neuronal cell culture bioreactor is equipped with three in-/outflow devices 11a, 11b, and 11c on three neighboring sides of the cube shaped container 4, each exhibiting a media supply 12a, 12b, respectively 12c.

Via the media supply a pump extracts media from the container 4 through a tube 16. The media is then enriched with oxygen (18), tempered in a heat exchanger and then returned to the container 4 through the inflow device opposite of the container 4. The supply or extraction can occur either via the hollow fiber structures 5c or via porosities in the container walls containing the in-/outflow devices 11c, respectively in-/outflow devices on the opposite container wall.

The in-/outflow devices 11a and 11b serve for the supply and removal of additional substances.

FIG. 7 shows a photography of a bioreactor as it is described in FIG. 6. A schematic drawing is included to better demonstrate the features. The same reference markers indicate the same elements, which render an explanation of the neuronal cell culture bioreactor 1 (generated from plexiglas container elements and polyamide hollow fiber membranes).

1. Neuronal cell culture bioreactor (1) for the culture, proliferation, differentiation, and/or of neuronal cells (2) in a container (4), as well as at least two from each other independent hollow fiber membrane systems (or channel like pathways) (5a, 5b, 5c, 5d) with a multitude of individual hollow fibers that intersect inside the container (4) and/or overlay each other, each leading in and/or out of the container (4); whereby the individual hollow fibers (5a through 5d) are equipped to conduct electrical signals and/or lead neuronal cell projections (10) in their lumen.

2. Neuronal cell culture bioreactor (1) according to above mentioned claim is thereby characterized that a multitude of neuronal cells (2) are arranged in the container (4) in such a way that the cell bodies, possibly with the exception of the neuronal projection, are located between the hollow fiber membrane systems (5a through 5d).

3. Neuronal cell culture bioreactor (1) according to one of afore mentioned claims is thereby characterized that at least one hollow fiber membrane system (5a through 5d) exhibits numerous parallel running hollow fibers (or channel like pathways).

4. Neuronal cell culture bioreactor (1) according to one of afore mentioned claims is thereby characterized that at least three independent hollow fiber membrane systems (5a through 5d) are present inside the container (4).

5. Neuronal cell culture bioreactor (1) according to one of the afore mentioned claims is thereby characterized that the hollow fibers (5a through 5d) intersect and/or overlay each other in all three directions/dimensions.

6. Neuronal cell culture bioreactor (1) according to one of the afore mentioned claims is thereby characterized that at least three hollow fiber membrane systems (5a through 5d) are provided whose hollow fibers each extend along side the direction of one of the Cartesian coordinates.

7. Neuronal cell culture bioreactor (1) according to one of the afore mentioned claims is thereby characterized that the sections of the hollow fiber walls (5a through 5d) inside the container (4) exhibit at least partially and/or in certain sections openings (6), e.g. micro pore like openings.

8. Neuronal cell culture bioreactor (1) according to the afore mentioned claim is thereby characterized that the sections of the hollow fiber walls (5a through 5d) inside the container (4) exhibit at least partially and/or in certain sections openings (6) with a diameter <1 micrometer.

9. Neuronal cell culture bioreactor (1) according to one of the afore mentioned claims is thereby characterized that the sections of the hollow fiber walls (5a through 5d) inside the container (4) exhibit at least partially and/or in certain sections openings (6) with a diameter between 1-5 micrometer.

10. Neuronal cell culture bioreactor (1) according to one of afore mentioned claims is thereby characterized that the hollow fibers (5a through 5d) exhibit at least in part electrical conductivity inside their lumen.

11. Neuronal cell culture bioreactor (1) according to one of afore mentioned claims is thereby characterized that the hollow fibers (5a through 5d) are at least partially equipped with an electrical conductor (9) on the inside, respectively an electrode, or that the hollow fiber itself is replaced with and electrical conductor, respectively and electrode.

12. Neuronal cell culture bioreactor (1) according to one of afore mentioned claims is thereby characterized that the hollow fibers (5a through 5d) contain at least partially an electrolyte, for example in form watery media or gel.

13. Neuronal cell culture bioreactor (1) according to one of afore mentioned claims is thereby characterized that the cell projections (10) of the neuronal cells (2) at least partially penetrate the hollow fibers (5a through 5d), are arranged inside the hollow fibers (5a through 5d), or extend along side the hollow fibers inside the hollow fibers (5a through 5d).

14. Neuronal cell culture bioreactor (1) according to one of the afore mentioned claims is thereby characterized that one or several of the hollow fibers (5a through 5d) are connected with signal transmitters and/or sensors for the transmission of electrical signals generated inside the signal transmitters or sensors, and/or with electrical receptors and/or effectors for the reception of electrical signals in the respective hollow fiber (5a through 5d).

15. Neuronal cell culture bioreactor (1) according to afore mentioned claims is thereby characterized that one or multiple hollow fibers (5a through 5d) are connected to a computer system via sensors, signal transmitters, signal receptors, and/or effectors.

16. Neuronal cell culture bioreactor (1) according to one of the afore mentioned claims is thereby characterized that the outer side of the container (4) exhibits at least one in-/outflow device (11a through 11c) for the supply of the container (4) and/or the hollow fiber (5a through 5d), for example with culture medium, nutrients, oxygen, electrolytes mediators, growth factors, hormones and such.

17. Neuronal cell culture bioreactor (1) according to one of the afore mentioned claims is thereby characterized that the outer side of the container (4) exhibits at least one in-/outflow device (11a through 11c) for the removal of e.g. biological metabolic waste produces from the container (4) and/or the hollow fibers (5a through 5d).

18. Neuronal cell culture bioreactor (1) according to one of the afore mentioned claims is thereby characterized that at least one in-/outflow device (11a through 11c) communicates with the container (4) or the hollow fibers (5a through 5d) that penetrate the container.

19. Neuronal cell culture bioreactor (1) according to one of the afore mentioned claims is thereby characterized that hollow fibers of at least one hollow fiber membrane system (5a through 5d) meet with at least one in-/outflow device (11a through 11c).

20. Neuronal cell culture bioreactor (1) according to one of afore mentioned claims is thereby characterized that the in-/outflow devices (11a through 11c) are connected with the container (4) and/or are an integral part of the container (4).

21. Neuronal cell culture bioreactor (1) according to one of afore mentioned claims is thereby characterized that at least one hollow fiber (5a through 5d) outside the container (4) is surrounded with a sheathing (13, 19).

22. Neuronal cell culture bioreactor (1) according to afore mentioned claims is thereby characterized that the sheathing (13, 19) is electrically insulating.

23. Neuronal cell culture bioreactor (1) according to one of the two afore mentioned claims is thereby characterized that the sheathing (13, 19) and at least one hollow fiber (5a through 5d) surrounded by sheathing exhibit a hollow space.

24. Neuronal cell culture bioreactor (1) according to one of the three afore mentioned claims is thereby characterized that the hollow space (5a through 5d) surrounded by a sheathing exhibits openings through which the lumen of the hollow spaces can be supplied and/or disposed of substances.

25. Neuronal cell culture bioreactor (1) according to one of the four afore mentioned claims is thereby characterized that the sheathing (19) is a perfusion tube.

26. Neuronal cell culture bioreactor (1) according to one of afore mentioned claims is thereby characterized that at least one of the hollow fibers exhibits a coating of the hollow fiber membrane with biomatrix.

27. Neuronal cell culture bioreactor (1) according to one of afore mentioned claims is thereby characterized that the container (4) contains a body (3) made of open porous material whose pores communicate with each other.

28. Neuronal cell culture bioreactor (1) according to one of the afore mentioned claims is thereby characterized that individual hollow channel like pathways of at least two pathway systems (5a through 5d) at least partially cross and/or overlay each other inside the body (3).

29. Neuronal cell culture bioreactor (1) according to one of the afore mentioned claims is thereby characterized that the hollow pathways of the body (3) exhibit a diameter of 5-1000 micrometer, preferably 50-100 micrometer.

30. Neuronal cell culture bioreactor (1) according to one of the afore mentioned claims is thereby characterized that connective tissue cells are arranged in co-culture inside the container (4) and/or the body (3) made from open porous material.

31. Neuronal cell culture bioreactor (1) according to one of afore mentioned claims is thereby characterized that animal or human adult neuronal cells, neuronal progenitor stem cells, glia cells, stroma cells, feeder cells, neuronal cell lines, gene-technologically modified cells, and/or embryonic stem cells are arranged inside the container (4) and/or the body (3).

32. Neuronal cell culture bioreactor arrangements under the utilization of neuronal cell culture bioreactors (1a, 1b) according to one of the afore mentioned claims are thereby characterized that at least two neuronal cell culture bioreactors (1a, 1b) are connected according to one of the above mentioned claims in such a way that one or several hollow fibers of at least one hollow fiber membrane system (5a through 5d) leads out of the container (4a) of the first neuronal cell culture bioreactor (1a), with one or several hollow fibers of at least one hollow fiber membrane system (5e through 5g) that leads out of the second container (4b) of a second neuronal cell culture bioreactor (1b), for the transmission of electrical signals or direction of neuronal cell projections (10), who are connected to their lumen, whereby at least one computer system with signal reception/processing/transmission/effectors can be arranged with the bioreactors.

33. Neuronal cell culture bioreactor (1) according to the afore mentioned claim is thereby characterized that the hollow fibers of a hollow fiber membrane system (5a through 5g), extending between two neuronal cell culture bioreactors (1a, 1b), are arranged in a hollow fiber bundle, which may be sheeted and perfused with culture medium.

34. Utilization of a neuronal cell culture bioreactor (1, 1a, 1b) or an arrangement of neuronal cell culture bioreactors according to one of the afore mentioned claims for the culture, co-culture, compartmentalized co-culture, proliferation and/or differentiation of neuronal cells.

35. Utilization of the invention according to the afore mentioned claim for the exploration of the functions of neuronal structures, therapy of diseases related to the nervous system or brain, in regenerative medicine and/or information processing, and/or signal processing, and signal transformation.

36. Utilization according to afore mentioned claims for the production of cell transplants, for example from stem cells/progenitor cells, for regenerative medicine.

37. Utilization according to the afore mentioned claims for the industrial use as hybrid nervous system or parts thereof, and/or for information processing, and/or signal processing/transformation.
